(19) United States
(12) Patent Application Publication
(10) Pub. No.: US 2026/0216065 A1
(43) Pub. Date: Jul. 30, 2026

(54) PHARMACEUTICAL COMPOSITIONS FOR NASAL DELIVERY

(71) Applicant: Therakind Limited, London (GB)

(72) Inventors: Julie-Ann Penton, London (GB); Susan Conroy, London (GB); Louise Rawcliffe, London (GB)

(73) Assignee: Therakind Limited, London (GB)

(21) Appl. No.: 19/151,721

(22) PCT Filed: Jan. 30, 2024
(86) PCT No.: PCT/GB2024/050251

§ 371 Date: Jul. 29, 2025

(30) Foreign Application Priority Data
Jan. 30, 2023 (GB) 2301322.0

(51) Int. Cl. A61K 9/00; A61K 9/16; A61K 31/166; A61K 31/40; A61K 31/4178; A61K 31/4192; A61K 31/437; A61K 31/439; A61K 31/4515; A61K 31/454; A61K 31/46; A61K 31/473; A61K 31/5415; A61K 31/551; A61P 1/08

(52) U.S. Cl. CPC A61K 9/0075; A61K 9/1623; A61K 9/1641; A61K 9/1652; A61K 31/166; A61K 31/40; A61K 31/4178; A61K 31/4192; A61K 31/437; A61K 31/439; A61K 31/4515; A61K 31/454; A61K 31/46; A61K 31/473; A61K 31/5415; A61K 31/551; A61P 1/08

(57) ABSTRACT

There is described a dry powder pharmaceutical composition for intranasal delivery, wherein the composition comprises a therapeutically effective amount of a medicament for the treatment of a disorder of the alimentary system, in free form or in salt form; one or more mucoadhesives; and optionally one or more pharmaceutically acceptable excipients.

### FIELD OF THE INVENTION

The present invention relates to compositions for nasal delivery for the treatment of a disorder of the alimentary canal, and methods of treatment related thereto.

More particularly, the invention relates to dry powder compositions for nasal delivery for the treatment of a disorder of the alimentary canal, including antiemetic therapies and therapies for the treatment of a gastrointestinal disorder, wherein said compositions include a mucoadhesive; and methods of treatment related thereto.

Antiemetic therapies include, for example, 5-HT₃ receptor antagonists, dopamine antagonists, NK1 receptor antagonists, antihistamines, cannabinoids, benzodiazepines, anticholinergics and steroids.

### BACKGROUND TO THE INVENTION

The known medicaments for treatment of a disorder of the alimentary system, including emesis, have some drawbacks in terms of limited efficacy, tolerability and convenience.

Antiemetic therapies are used to treat, prevent or alleviate nausea and vomiting, to help with emptying of the stomach in patients with delayed stomach emptying, to help with gastroesophageal reflux disease, motion sickness, the side effects of opioid analgesics, the side effects of general anaesthetics and to alleviate the symptoms of migraine headaches.

Known side effects of antiemetic medicaments include constipation, diarrhoea, dry mouth, fatigue and dizziness.

There are various classes of antiemetic medicaments, including 5-HT₃ receptor antagonists, dopamine antagonists, NK1 receptor antagonists, antihistamines, cannabinoids, benzodiazepines, anticholinergics and steroids.

One of the most used classes of compounds is the 5-HT₃ receptor antagonists, also known as the “setrons”, which include, for example, granisetron, ondansetron and palonosetron.

Ondansetron is (RS)-9-Methyl-3-[(2-methyl-1H-imidazol-1-yl)methyl]-2,3-dihydro-1H-carbazol-4(9H)-one.

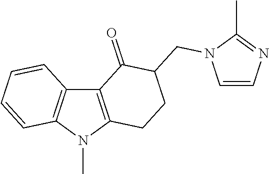 [SMILES: Cc1nccn1CC1CCc2c(c3ccccc3n2C)C1=O]

Setrons, such as, granisetron, ondansetron and palonosetron, are highly selective 5-HT₃ receptor antagonists and are used for the prevention of nausea and vomiting associated with cancer chemotherapy, radiation therapy or surgery. Nausea and vomiting can lead to dehydration, severe metabolic imbalance and malnutrition in patients.

Setrons are generally administered orally as a tablet or an orally disintegrating tablet, or by injection into a muscle or vein. They are usually administered orally in doses ranging from 8 to 24 mg, as a single daily dose or as a pre- and post-chemotherapy dose.

Intravenous setrons are administered at a lower dose of usually about 40-80 mg/kg for adults for the prevention of nausea and vomiting associated with initial and repeat courses of emetogenic cancer chemotherapy and at doses of about 4 mg for prevention of postoperative nausea and vomiting.

Ondansetron is widely prescribed, in 2020, it was the 83rd most commonly prescribed medication in the United States, with more than 8 million prescriptions.

Ondansetron, together with its physiologically acceptable salts and solvates, is described in United Kingdom Patent No. GB2153821, and may be used in the treatment of a variety of conditions, including the treatment of nausea and vomiting induced by cancer chemotherapy and radiotherapy.

Other antiemetics that may be mentioned include the dopamine antagonists, such as olanzapine and metoclopramide.

However, whilst conventional oral administration provides good systemic absorption of ondansetron, it takes a significant time to enter a patient's system and bring relief. Furthermore, the nausea and vomiting associated with cancer chemotherapies, often cause the cancer therapy and the antiemetic ondansetron to be discharged by vomiting. Similar problems can arise with post-operative nausea and vomiting.

With intravenous administration a patient experiences undesirable side effects, including, constipation, diarrhoea, and dizziness.

Administering lower doses of antiemetics will generally give rise to fewer side effects. Recent developments have included administration of lower doses of antiemetics, such as ondansetron, by nasal spray.

US Patent application No. 2014/303221 describes a composition comprising ondansetron for intranasal administration in combination with an antimicrobial preservative and a long chain polymer enhancing the viscosity of the composition.

US Patent application No. 2003/044356 describes a composition comprising ondansetron and a base material, said base material comprising 70-85% water, 5-15% polyethylene glycol, 0.005-0.02% benzalkonium chloride and 7-20% a solubiliser selected from sulfobutyl ether β-cyclodextrin sodium salt, dimethyl-β-cyclodextrin and 2-hydroxypropyl-β-cyclodextrin.

US Patent application No. 2015/010633 describes formulation for nasal delivery comprising an ondansetron and a propellant.

US Patent application No. 2016/206610 describes a method of treating acute nausea and/or acute emesis comprising administering a 5-HT₃ antagonist by nasal inhalation and/or oral inhalation.

Oral or nasal delivery of a medicament in dry powder form, e.g. using a delivery device, is a particularly attractive method of drug administration as such devices can be relatively easy for a patient or non-medically qualified carer to use.

However, nasal spray solutions of ondansetron are generally only stable for 48 hours at room temperature (25° C.). Therefore, ondansetron compositions in the form of dry powders for inhalation offer advantages over other forms, such as liquids, e.g. nasal spray liquids, particularly when considering storage and stability; and the capabilities of patients and their carers.

Advantages of delivering ondansetron or other medications for the treatment of a disorder of the alimentary system, including antiemetic medications, in a dry powder form is that the onset of action can be rapid, first pass metabolism is avoided, bioavailability may be improved and the need for preservatives may be mitigated.

Therefore, there is a need for a dry powder form of medications, for the treatment of a disorder of the alimentary system, including antiemetic medications, especially for use in nasal delivery.

### SUMMARY OF THE INVENTION

According to a first aspect of the invention there is provided a dry powder pharmaceutical composition for intranasal delivery, wherein the composition comprises a therapeutically effective amount of a medicament for the treatment of a disorder of the alimentary system, in free form or in salt form; one or more mucoadhesives; and optionally one or more pharmaceutically acceptable excipients.

According to one aspect of the invention the medicament may comprise a medicament for the treatment of emesis, i.e. an antiemetic medicament.

According to another aspect of the invention the medicament may comprise a medicament for the treatment of a disorder of the gastrointestinal system.

The medicament for the treatment of a disorder of the alimentary system may be selected from the group comprising 5-HT₃ receptor antagonists, dopamine antagonists, NK1 receptor antagonists, antihistamines, cannabinoids, benzodiazepines, anticholinergics, including antispasmodics, steroids, opioid receptor agonists, opioid receptor antagonists (including mixed agonists/antagonists), antibiotics, chloride channel activators, guanylate cyclase-C receptor agonists, antidiarrheals, in free form or in salt form; and combinations thereof.

In a preferred aspect of the invention the medicament for the treatment of a disorder of the alimentary system may be selected from the group comprising 5-HT₃ receptor antagonists, dopamine antagonists, NK1 receptor antagonists, antihistamines, cannabinoids, benzodiazepines, anticholinergics and steroids, in free form or in salt form; and combinations thereof.

Suitable 5-HT₃ receptor antagonists is a setron which includes, but shall not be limited to, one or more of dolasetron, granisetron, ondansetron, tropisetron, alosetron or palonosetron, in free form or in salt form or a derivative thereof, and combinations thereof. One 5-HT₃ receptor antagonist which may be mentioned is ondansetron, in free form or in salt form or a derivative thereof. A further 5-HT₃ receptor antagonist which may be mentioned is granisetron, in free form or in salt form or a derivative thereof. A yet further 5-HT₃ receptor antagonist which may be mentioned is palonosetron, in free form or in salt form or a derivative thereof.

Suitable dopamine antagonists include, but shall not be limited to, one or more of amisulpride, domperidone, olanzapine, haloperidol, alizapride, prochlorperazine, chlorpromazine or metoclopramide, in free form or in salt form or a derivative thereof, and combinations thereof. A particular dopamine antagonist which may be mentioned is metoclopramide, in free form or in salt form or a derivative thereof. A further dopamine antagonist which may be mentioned is olanzapine, in free form or in salt form or a derivative thereof.

Suitable NK1 receptor antagonists, but shall not be limited to, one or more of aprepitant, casopitant or rolapitant, in free form or in salt form or a derivative thereof; and combinations thereof.

Suitable antihistamines include, but shall not be limited to, one or more of cinnarizine, cyclizine, diphenhydramine, dimenhydrinate, doxylamine, mirtazapine, meclizine, promethazine or hydroxyzine, in free form or in salt form or a derivative thereof, and combinations thereof.

Suitable cannabinoids include, but shall not be limited to, one or more of nabilone or dronabinol, in free form or in salt form or a derivative thereof, and combinations thereof.

Suitable benzodiazepines include, but shall not be limited to, one or more of midazolam or lorazepam, in free form or in salt form or a derivative thereof, and combinations thereof.

Suitable anticholinergics include, but shall not be limited to, one or more of scopolamine or atropine, in free form or in salt form or a derivative thereof, and combinations thereof.

Suitable steroids include, but shall not be limited to, dexamethasone, in free form or in salt form or a derivative thereof.

The pharmaceutical composition as herein described may be suitable for intranasal delivery using a powder inhaler, insufflator, powder sprayer or nebuliser. Hereinafter the term “inhaler” should be construed as meaning a powder inhaler, insufflator, powder sprayer or nebuliser

In the dry powder pharmaceutical composition of the invention, the primary particle size distribution is determined via dispersion of the bulk powder at high pressure and measurement of the primary particle size distribution via laser diffraction. “Primary particles” refer to the individual particles that are present in an agglomerated bulk powder i.e. the therapeutically active antiemetic agent. The primary particles in the powder composition, will generally have a mean diameter of >10 μm, that is a substantial portion of the primary particles have a mean diameter of >10 μm and a D50 of from about 20 to 60 μm. This means that, 50 percent of the particles measure from about 20 to 60 μm. Preferably, the powder composition will generally have a D₅₀ of from about 25 to 55 μm, more preferably from about 30 to 50 μm. The smaller particles are generally considered undesirable, therefore, the number of particles with a diameter of 10 μm or less will be ≤20% w/w, preferably ≤15% w/w, more preferably ≤10% w/w, most preferably ≤10% w/w.

Suitable pharmaceutically acceptable excipients shall include carriers that may be incorporated in the pharmaceutical composition. The pharmaceutically acceptable excipients may vary depending, inter alia, upon the nature of the medicament. Such carriers include, but shall not be limited to, sugars such as glucose, saccharose, lactose and fructose, saccharides, disaccharides, amino acids such as but not limited to, glycine, leucine, isoleucine, arginine, starches or starch derivatives, oligosaccharides such as dextrins, cyclodextrins and their derivatives, such as 2-hydroxypropyl-β-cyclodextrin, polyvinylpyrrolidone, alginic acid, tylose, silicic acid, organic salts such as but not limited to, sodium citrate, ammonium acetate, cellulose, including microcrystalline cellulose and colloidal microcrystalline cellulose; cellulose derivatives, for example cellulose ether, calcium carbonate, calcium phosphate, lactose, lactitol, dextrates, calcium stearate, dextrose, maltodextrin, saccharides including monosaccharides, disaccharides; polysaccharides, such as carrageenan; sugar alcohols such as mannitol, sorbitol, arabinose, ribose, mannose, sucrose, trehalose, maltose, dextran; magnesium stearate, cellobiose octaacetate and the like; and combinations thereof.

The dry powder composition will generally comprise a free flowing dry powder.

In one aspect of the invention the carrier may comprise a sugar. In a particular aspect of the invention the sugar carrier is selected from the group comprising one or more of glucose, saccharose, lactose and fructose, saccharides, disaccharides, sugar alcohols such as mannitol or sorbitol, lactose, lactitol, dextrates, dextrose, maltodextrin, saccharides including monosaccharides, disaccharides, polysaccharides; sugar alcohols such as arabinose, ribose, mannose, sucrose, trehalose, maltose, dextran. In another aspect of the invention the carrier may be an organic salt, such as a fatty acid salt, e.g. magnesium stearate.

In a particular aspect of the invention the carrier is selected from the group consisting of one or more of mannitol, trehalose and lactose. In one aspect of the invention the carrier comprises mannitol. In another aspect of the invention the carrier comprises trehalose. In another aspect of the invention the carrier comprises lactose.

The amount of sugar carrier may vary, but the sugar carrier may be present in an amount of from about 60% w/w to about 90% w/w (based on the weight of excipients), preferably from about 60% w/w to about 85% w/w, preferably from about 65% w/w to about 85% w/w, preferably from about 65% w/w to about 80% w/w, preferably from about 65% w/w to about 80% w/w, preferably from about 70% w/w to about 80% w/w. About 70% w/w sugar carrier is most preferred.

The one or more mucoadhesives may comprise a mucoadhesive polymer, i.e. an agent that improves residence time of the composition in the nose. Any suitable mucoadhesive may be used. Suitable mucoadhesive polymers is selected from, but shall not be limited to, alkyl celluloses, such as methylcellulose, hydroxyalkyl celluloses, such as hydroxypropyl methylcellulose (HMPC) and alginates, i.e. esters and salts of alginic acid, polyacrylic acids, such as carbopol, gelatin, gellan gum, polyethylene glycols, pectin, chitosans; and combinations thereof. One preferred mucoadhesive is methylcellulose. Another preferred mucoadhesive is hydroxypropyl methylcellulose (HMPC). The amount of mucoadhesive may vary, but the mucoadhesive may be present in an amount of from about 10% w/w to about 40% w/w (based on the weight of excipients), preferably from about 15% w/w to about 40% w/w, preferably from about 15% w/w to about 35% w/w, preferably from about 20% w/w to about 35% w/w, preferably from about 20% w/w to about 30% w/w, preferably from about 25% w/w to about 30% w/w. About 30% w/w mucoadhesive is most preferred.

A preferred composition of the invention may comprise a combination of a mucoadhesive and a carrier, e.g. a sugar. According to this aspect of the invention a preferred composition comprises about 30% w/w (based on the weight of excipients) of a mucoadhesive, such as HPMC; and about 70% w/w of a sugar carrier, such as mannitol.

Another preferred composition may comprise a combination of a mucoadhesive and an absorption enhancer, such as a cyclodextrin carrier. Cyclodextrins act as a filler, and also adhere to the nasal mucosal surface and aid in the absorption of the medicament. Upon delivery to the nostril, the major ingredient (90% to 70% by weight) namely, the cyclodextrin helps the powder adhere to the mucosal surface. The less soluble the cyclodextrin is, the longer the powder is expected to be physically mucoadhesive. Cyclodextrins may be used individually, or as mixtures of any two or more cyclodextrins. The amount of cyclodextrin present may vary and may be from about 40% w/w to about 90% w/w of cyclodextrin. It will be understood that other absorption enhancers may be used either separately or in addition to a cyclodextrin. Suitable absorption enhancers may comprise surfactants e.g. polysorbates, such as, polysorbate 20 and/or polysorbate 80. According to this aspect of the invention composition may comprise a combination of a mucoadhesive and an absorption enhancer, such as a cyclodextrin carrier. A preferred composition comprises about 30% w/w (based on the weight of excipients) of a mucoadhesive, such as HPMC; and about 70% w/w of a cyclodextrin carrier.

The composition of the invention may include one or more absorption enhancers. Absorption enhancers may improve the bioavailability of the administered medicament. Any suitable absorption enhancers may be used. Suitable absorption enhancers are selected from, but shall not be limited to, cyclodextrins, phospholipids, surfactants, such as polysorbates, chitosan, bile salts, fatty acids and chelators; and combinations thereof. One or more cyclodextrins are preferred absorption enhancers. In some embodiments of the invention the absorption enhancer may also act as a carrier. Suitable absorption enhancer that may also act as a carrier, i.e. may remove the need for a separate carrier, include, but shall not be limited to, a cyclodextrin, a polysorbate or a chitosan.

In the composition of the invention the medicament for the treatment of a disorder of the alimentary system may be present in free form or as a pharmaceutically acceptable salt or derivative thereof, and optionally combinations thereof.

As used herein, the term “salt” shall generally mean a “pharmaceutically acceptable salt” and refers to salts that retain the biological effectiveness and properties of the compounds of this invention and, which are not biologically or otherwise undesirable.

A pharmaceutically acceptable salt is any salt of the parent compound that is suitable for administration to an animal or human. A pharmaceutically acceptable salt also refers to any salt which may form in vivo as a result of administration of an acid, another salt, or a prodrug which is converted into an acid or salt. A salt comprises one or more ionic forms of the compound, such as a conjugate acid or base, associated with one or more corresponding counter-ions. Salts can form from or incorporate one or more deprotonated acidic groups (e.g. carboxylic acids) one or more protonated basic groups (e.g. amines), or both (e.g. zwitterions).

As used herein, the term “pharmaceutically acceptable salts” refers to salts that retain the biological effectiveness and properties of the compounds of this invention and, which are not biologically or otherwise undesirable. In many cases, the compounds of the present invention are capable of forming acid and/or base salts by virtue of the presence of amino and/or carboxyl groups or groups similar thereto. Pharmaceutically acceptable acid addition salts can be formed with inorganic acids and organic acids, e.g., acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts. Inorganic acids from which salts can be derived include, for example, hydrochloric acid, hydrobromic acid, sulphuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulphonic acid, ethanesulphonic acid, p-toluenesulphonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminium, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. The pharmaceutically acceptable salts of the present invention can be synthesized from a parent compound, a basic or acidic moiety, by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are preferred, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

When the medicament for the treatment of a disorder of the alimentary system is a setron, such as, ondansetron, it may be present as the free base or as a pharmaceutically acceptable salt or derivative thereof, and optionally combinations thereof. Suitable pharmaceutically acceptable salts of a setron, such as, ondansetron, include acid addition salts formed with organic or inorganic acids for example, hydrochlorides, hydrobromides, sulphates, phosphates, citrates, fumarates and maleates. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002). Ondansetron hydrochloride is especially preferred.

It will be understood by the person skilled in the art that the term “derivative” shall include, inter alia, pharmaceutically acceptable salts, solvates, esters and pro-drugs. Non-pharmaceutically acceptable salts, solvates, esters and pro-drugs should also be considered to fall within the scope of the present invention, since those may be useful in the preparation of pharmaceutically acceptable salts, solvates or pro-drugs.

The preparation of salts, solvates and pro-drugs can be carried out by methods known to the person skilled in the art. It will be appreciated that non-pharmaceutically acceptable salts, solvates or pro-drugs also fall within the scope of the invention since those may be useful in the preparation of pharmaceutically acceptable salts, solvates or pro-drugs. Methods of solvation are generally known within the art. Suitable solvates are pharmaceutically acceptable solvates.

According to a further aspect of the present invention there is provided the use of a medicament for the treatment of a disorder of the alimentary system in the manufacture of a dry powder pharmaceutical composition for treating, preventing or alleviating a disorder of the alimentary system. In particular, there is provided the use as herein described in the manufacture of a dry powder pharmaceutical composition for nasal administration.

Disorders of the alimentary system include, but shall not be limited to, emesis, gastrointestinal disorders, such as, gastroesophageal reflux disease (GERD), dysphagia, ulcer, irritable bowel syndrome (IBS), inflammatory bowel disease (IBD), gastroparesis, colitis and Crohn's disease.

Disorders of the alimentary system are well known to the person skilled in the art. These disorders include emesis, including nausea and vomiting, particularly that associated with cancer chemotherapy and radiotherapy and also that occurring post operatively.

In one aspect of the invention the pharmaceutical composition of the invention comprises a medicament for the treatment of emesis, i.e. an antiemetic medicament.

In another aspect of the invention the pharmaceutical composition of the invention comprises a medicament for the treatment of a disorder of the gastrointestinal system, such as, Gastro-Esophageal Reflux Disease (GERD), Irritable Bowel Syndrome (IBS) or gastroparesis.

For the avoidance of doubt, the gastrointestinal system shall mean the tract or passageway of the digestive system that leads from the mouth to the anus. The gastrointestinal system includes all the major organs of the digestive system in humans and other animals, including the oesophagus, stomach and intestines. Gastrointestinal should be given its conventionally understood meaning of or pertaining to the stomach and intestines.

Disorders of the gastrointestinal system shall include, but shall not be limited to, Gastro-Esophageal Reflux Disease (GERD), Functional Gastrointestinal Disorders and Post-operative Ileus. Functional Gastrointestinal Disorders (FGIDs) are defined as chronic or recurrent conditions associated with abdominal symptoms without organic cause using conventional diagnostic measures. A cardinal symptom present in many FGIDs is visceral pain and/or discomfort. FGIDs include Functional Dyspepsia (FD), functional heartburn (a subset of GERD), Irritable Bowel Syndrome (IBS), gastroparesis, Functional Bloating, Functional Diarrhoea, Chronic Constipation, Functional Disturbances of the Biliary Tract as well as other conditions according to Gut 1999; Vol. 45 Suppl. II.

Post-operative Ileus is defined as failure of aboral passage of intestinal contents due to transient impairment of GI motility following abdominal surgery.

Gastro-Esophageal Reflux Disease (GERD) results from the retrograde flow of gastric contents into the oesophagus. It is the most common ailment in the upper gastrointestinal tract; its cardinal feature and symptom is commonly known as “heartburn”. A major factor considered for GERD is an incompetence of the Lower Esophageal Sphincter that opens transiently and allows passage of material (e.g. meal, acidic fluid or bile), from the stomach into the oesophagus. This motor event denominated Transient Lower Esophageal Sphincter Relaxation (TLESR) occurs more often in patients suffering from GERD than in healthy subjects and occurs more often in infants with regurgitation. Current standard therapies in GERD aim at suppressing gastric acid secretion or enhancing gastrointestinal motility to limit the exposure of the oesophagus to acidic gastric contents. Frequent exposure of the oesophageal mucosa to acid can trigger pain (often perceived as heartburn) and lead to erosions. It can also lead to extra-oesophageal disorders such as asthma, cough and laryngitis.

Functional Dyspepsia (FD) is defined as a condition associated with a heterogeneous pattern of upper abdominal symptoms including discomfort, pain, aching, bloating, belching, fullness, early satiety, nausea and vomiting, burning and indigestion. Almost 80% of patients with Functional Dyspepsia have two or more of the above mentioned symptoms of the upper GI tract.

The pathophysiological abnormalities observed in FD are as follows:
Impaired gastric accommodation upon meal intake, hypersensitivity to gastric distension, delayed gastric emptying, autonomous and/or central nervous system disorder, exaggerated phasic contractile activity, abnormalities of the gastric electrical rhythm, duodenal hypersensitivity to lipids or acid, small intestinal dysmotility. Meals evoke symptoms in more than 75% of FD patients, and symptoms increase with meal ingestion in more than 90% of patients. Therefore, a treatment that prepares the stomach to meal intake has the potential to reduce meal-evoked symptoms. In fact, low fasting volume (not postprandial volume) was found to be an independent predictor for reduced meal size and post-meal symptoms in FD patients (Delgado-Aros et al., Gastroenterology, 2004; 127:1685-1694).

Irritable Bowel Syndrome (IBS) is a chronic or remittent gastrointestinal illness characterized by symptoms that include abdominal pain and/or discomfort, bloating and bowel disturbances, which may be either diarrhoea or constipation or a bowel habit that has features of both. Pain and/or Discomfort is often associated with FGIDs, disorders of the urinary tract and post-operative ileus; it is not only a symptom of GERD. Patients suffering from Irritable Bowel Syndrome (IBS), dyspepsia, diseases of the biliary tract, pancreas, urinary bladder and post-operative conditions report pain and discomfort. Visceral hypersensitivity has been discovered as a key phenomenon in many patients suffering from conditions like IBS, dyspepsia, GERD, functional heartburn and other conditions listed above. To date, there is no medicament available which specifically treats visceral hypersensitivity and, thereby, reduces symptoms of pain/discomfort in patients suffering from GERD, functional heartburn, IBS, dyspepsia, diseases of the biliary tract, pancreas, urinary bladder and post-operative conditions. Pain, as used in this specification, includes visceral pain and/or visceral discomfort.

According to a further aspect of the present invention there is provided the use of a setron, such as ondansetron, in free form or in salt form or a derivative thereof, in the manufacture of a dry powder pharmaceutical composition for treating, preventing or alleviating a gastrointestinal disorder, in particular, emesis. In particular, there is provided the use as herein described in the manufacture of a dry powder pharmaceutical composition for nasal administration.

According to a yet further aspect of the present invention there is provided the use of a composition as herein described in the manufacture of a dry powder pharmaceutical for treating, preventing or alleviating a disorder of the alimentary system, in particular, emesis or a gastrointestinal disorder. In particular, there is provided the use as herein described in the manufacture of a dry powder pharmaceutical composition for nasal administration, i.e. nasal inhalation or insufflation.

According to a further aspect of the present invention there is provided a method of treating, preventing or alleviating a disorder of the alimentary system, in particular, emesis or a gastrointestinal disorder, by administering a therapeutically effective amount of a pharmaceutical composition in dry powder form, said composition comprising a medicament for the treatment of a disorder of the alimentary system, in free form or in salt form or a derivative thereof, in dry powder form.

In particular, there is provided a method as herein described which comprises administering a therapeutically effective amount of a pharmaceutical composition comprising a medicament for the treatment of a disorder of the alimentary system, in free form or in salt form or a derivative thereof, in dry powder form by nasal administration.

According to one aspect of the invention the method comprises treating, preventing or alleviating emesis.

According to one aspect of the invention the method comprises treating, preventing or alleviating a conventional gastrointestinal disorder.

According to a yet further aspect of the present invention there is provided a method of treating, preventing or alleviating a disorder of the alimentary system, in particular, emesis or a gastrointestinal disorder, by administering a therapeutically effective amount of a pharmaceutical composition comprising a setron, such as ondansetron, in free form or in salt form or a derivative thereof, in dry powder form. In particular, there is provided a method as herein described which comprises administering a therapeutically effective amount of a pharmaceutical composition comprising a setron, such as ondansetron, in free form or in salt form or a derivative thereof, in dry powder form by nasal administration.

Conditions mediated through the action of 5-HT₃ receptor antagonists, dopamine antagonists, NK1 receptor antagonists, antihistamines, cannabinoids, benzodiazepines, anticholinergics and steroids, include nausea and vomiting, particularly that associated with cancer chemotherapy and radiotherapy and also that occurring post operatively; cognitive disorders such as dementia, particularly degenerative dementia (including senile dementia, Alzheimer's disease, Pick's disease, Huntington's chorea, Parkinson's disease and Creutzfeldt-Jakob disease), and vascular dementia (including multi-infarct dementia), as well as dementia associated with intracranial space occupying lesions, trauma, infections and related conditions (including HIV infection), metabolism, toxins, anoxia and vitamin deficiency; and mild cognitive impairment associated with ageing, particularly Age Associated Memory Impairment; psychotic disorders, such as schizophrenia and mania; anxiety disorders, including panic disorder, agoraphobia, social phobia, simple phobia, obsessive compulsive disorders, post-traumatic stress disorder, mixed anxiety and depression, and generalised anxiety disorder; irritable bowel syndrome and dependency on drugs and substances of abuse. Other conditions mediated in this manner include gastric stasis; symptoms of gastrointestinal dysfunction such as occur with peptic ulcer, reflux oesophagitis, flatulence and dyspepsia; migraine; obesity and conditions such as bulimia; pain; and depression.

The pharmaceutical composition of the present invention has particular utility for the treatment of a disorder of the alimentary system including emesis, gastrointestinal disorders, including, but not be limited to, gastroesophageal reflux disease (GERD), dysphagia, ulcer, irritable bowel syndrome (IBS), gastroparesis, inflammatory bowel disease (IBD), colitis and Crohn's disease.

When the composition of the invention is the treatment of emesis, this may include nausea and vomiting, especially that associated with cancer chemotherapy and radiotherapy, but also that occurring post-operatively.

It will be appreciated that the precise therapeutic dose of the active ingredient, i.e. the medicament for the treatment of a disorder of the alimentary system, will depend on the age and condition of the patient and the nature of the condition to be treated and will be at the ultimate discretion of the attendant physician.

However, in general, effective doses for the treatment of conditions mediated through the action of 5-HT₃ receptor antagonists, dopamine antagonists, NK1 receptor antagonists, antihistamines, cannabinoids, benzodiazepines, anticholinergics or steroids, for example post-operative or chemotherapy induced nausea and vomiting, will be from about 1 to about 12 mg (based on dose of medicament), preferably from about 2 to about 10 mg, more preferably from about 4 to about 8 mg, of the medicament for the treatment of a disorder of the alimentary system per unit dose.

According to another aspect of the present invention, there is provided a process of preparing a pharmaceutical composition in dry powder form comprising a medicament for the treatment of a disorder of the alimentary system, in free form or in salt form or a derivative thereof; one or more mucoadhesives; and optionally one or more pharmaceutically acceptable excipients.

According to another aspect of the present invention, there is provided a process of preparing a pharmaceutical composition in dry powder form comprising a setron, such as ondansetron, in free form or in salt form or a derivative thereof; one or more mucoadhesives; and optionally one or more pharmaceutically acceptable excipients.

The dry powder of the present invention may be obtained by any suitable process such as, but not limited, to milling, precipitation, homogenization, high pressure homogenization, spray-freeze drying, supercritical fluid technology, double emulsion/solvent evaporation, PRINT (Particle Replication In Non-wetting Templates), thermal condensation, ultrasonication, spray drying. Preferably, the dry powder will be spray dried or freeze dried or a combination thereof. Spray dried particles are generally amorphous and such amorphous particles are preferred.

According to a further aspect of the invention there is provided a dry powder nasal inhaler containing a medicament for the treatment of a disorder of the alimentary system, in dry powder form, in free form or in salt form or a derivative thereof; one or more mucoadhesives; and optionally one or more pharmaceutically acceptable excipients.

In particular there is provided a dry powder nasal inhaler containing a setron, such as ondansetron in dry powder form, in free form or in salt form or a derivative thereof; and one or more mucoadhesives.

Suitable dry powder inhalers will be known to the person skilled in the art. However, exemplary dry powder nasal inhalers include, but shall not be limited to, those disclosed in International patent application No. PCT/GB2012/000907 (WO 2013/088112) and International patent application No. PCT/GB2018/052453 (WO 2019/043390).

“Effective amount” means the amount of a compound that, when administered to a subject for the prophylaxis or treatment of a disease and/or condition, is sufficient to affect such prophylaxis or such treatment for the disease and/or condition. The “effective amount” can vary depending on the compound, the disease and/or condition and its severity, and the age, weight, etc., of the subject.

“Preventing” or “prevention” refers to a reduction in risk of acquiring or developing a disease and/or condition (i.e., causing at least one of the clinical symptoms of the disease and/or condition not to develop in a subject that may be exposed to a disease and/or condition-causing agent, or predisposed to the disease and/or condition in advance of disease and/or condition onset).

The term “prophylaxis” is related to “prevention”, and refers to a measure or procedure the purpose of which is to prevent, rather than to treat or cure a disease.

“Treating” or “treatment” of any disease and/or condition refers, in one embodiment, to ameliorating the disease and/or condition (i.e., arresting the disease or reducing the manifestation, extent or severity of at least one of the clinical symptoms thereof). In another embodiment “treating” or “treatment” refers to ameliorating at least one physical parameter, which may not be discernible by the subject. In yet another embodiment, “treating” or “treatment” refers to modulating the disease and/or condition, either physically, (e.g., stabilization of a discernible symptom), physiologically, (e.g., stabilization of a physical parameter), or both. In a further embodiment, “treating” or “treatment” relates to slowing the progression of the disease and/or condition.

The present invention will now be described by way of example only.

### EXAMPLE 1

### Excipients and Formulations

Excipients to be selected from mucoadhesives e.g. HPMC, sugar carriers e.g. mannitol, absorption enhancers e.g. HP-β-cyclodextrin and surfactants e.g. Polysorbate 80 as exemplified in Table 1.

TABLE 1
Antiemetic formulations

| API Dose, | | | |
|---|---|---|---|
| Loading | Excipient Loading | | |
| Ondansetron | HPMC 22% | Mannitol 51% | |
| 8 mg, 27% | HPMC 22% | Trehalose 51% | |
| | HPMC 22% | Lactose 51% | |
| | Methylcellulose 22% | Mannitol 51% | |
| | Methylcellulose 22% | Trehalose 51% | |
| | Methylcellulose 22% | Lactose 51% | |
| | HPMC 22% | HP-β-cyclodextrin 51% | |
| | HPMC 22% | β-cyclodextrin 51% | |
| | HPMC 22% | dM-β-cyclodextrin 51% | |
| | Methylcellulose 22% | HP-β-cyclodextrin 51% | |
| | Methylcellulose 22% | β-cyclodextrin 51% | |
| | Methylcellulose 22% | dM-β-cyclodextrin 51% | |
| | HPMC 19% | Mannitol 44% | HP-β-cyclodextrin |
| | | | 10% |
| | HPMC 19% | Mannitol 44% | β-cyclodextrin 10% |
| | HPMC 19% | Mannitol 44% | dM-β-cyclodextrin |
| | | | 10% |
| | HPMC 19% | Trehalose 44% | HP-β-cyclodextrin |
| | | | 10% |
| | HPMC 19% | Trehalose 44% | β-cyclodextrin 10% |
| | HPMC 19% | Trehalose 44% | dM-β-cyclodextrin |
| | | | 10% |
| | HPMC 19% | Lactose 44% | HP-β-cyclodextrin |
| | | | 10% |
| | HPMC 19% | Lactose 44% | β-cyclodextrin 10% |
| | HPMC 19% | Lactose 44% | dM-β-cyclodextrin |
| | | | 10% |
| | Methylcellulose | Mannitol 44% | HP-β-cyclodextrin |
| | 19% | | 10% |
| | Methylcellulose | Mannitol 44% | β-cyclodextrin 10% |
| | 19% | | |
| | Methylcellulose | Mannitol 44% | dM-β-cyclodextrin |
| | 19% | | 10% |
| | Methylcellulose | Trehalose 44% | HP-β-cyclodextrin |
| | 19% | | 10% |
| | Methylcellulose | Trehalose 44% | β-cyclodextrin 10% |
| | 19% | | |
| | Methylcellulose | Trehalose 44% | dM-β-cyclodextrin |
| | 19% | | 10% |
| | Methylcellulose | Lactose 44% | HP-β-cyclodextrin |
| | 19% | | 10% |
| | Methylcellulose | Lactose 44% | β-cyclodextrin 10% |
| | 19% | | |
| | Methylcellulose | Lactose 44% | dM-β-cyclodextrin |
| | 19% | | 10% |
| | HPMC 22% | Mannitol 50% | Polysorbate 80 1% |
| | HPMC 22% | Mannitol 50% | Polysorbate 20 1% |
| | HPMC 22% | Trehalose 50% | Polysorbate 80 1% |
| | HPMC 22% | Trehalose 50% | Polysorbate 20 1% |
| | HPMC 22% | Lactose 50% | Polysorbate 80 1% |
| | HPMC 22% | Lactose 50% | Polysorbate 20 1% |
| | Methylcellulose | Mannitol 50% | Polysorbate 80 1% |
| | 22% | | |
| | Methylcellulose | Mannitol 50% | Polysorbate 20 1% |
| | 22% | | |
| | Methylcellulose | Trehalose 50% | Polysorbate 80 1% |
| | 22% | | |
| | Methylcellulose | Trehalose 50% | Polysorbate 20 1% |
| | 22% | | |
| | Methylcellulose | Lactose 50% | Polysorbate 80 1% |
| | 22% | | |
| | Methylcellulose | Lactose 50% | Polysorbate 20 1% |
| | 22% | | |
| | Chitosan 20% | Mannitol 53% | |
| | Chitosan 20% | Trehalose 53% | |
| | Chitosan 20% | Lactose 53% | |
| | HPMC 16% | Mannitol 37% | Dodecylphoshocholine |
| | | | 20% |
| | HPMC 16% | Trehalose 37% | Dodecylphoshocholine |
| | | | 20% |
| | HPMC 16% | Lactose 37% | Dodecylphoshocholine |
| | | | 20% |
| | Methylcelluose | Mannitol 37% | Dodecylphoshocholine |
| | 16% | | 20% |
| | Methylcelluose | Trehalose 37% | Dodecylphoshocholine |
| | 16% | | 20% |
| | Methylcelluose | Lactose 37% | Dodecylphoshocholine |
| | 16% | | 20% |
| Metoclopramide | HPMC 20% | Mannitol 47% | |
| or Domperidone | HPMC 20% | Trehalose 47% | |
| 10 mg, 33% | HPMC 20% | Lactose 47% | |
| | Methylcellulose | Mannitol 47% | |
| | 20% | | |
| | Methylcellulose | Trehalose 47% | |
| | 20% | | |
| | Methylcelluose | Lactose 47% | |
| | 20% | | |
| | HPMC 20% | HP-β-cyclodextrin | |
| | | 47% | |
| | HPMC 20% | β-cyclodextrin 47% | |
| | HPMC 20% | dM-β-cyclodextrin | |
| | | 47% | |
| | Methylcellulose | HP-β-cyclodextrin | |
| | 20% | 47% | |
| | Methylcellulose | β-cyclodextrin 47% | |
| | 20% | | |
| | Methylcellulose | dM-β-cyclodextrin | |
| | 20% | 47% | |
| | HPMC 17% | Mannitol 40% | HP-β-cyclodextrin |
| | | | 10% |
| | HPMC 17% | Mannitol 40% | β-cyclodextrin 10% |
| | HPMC 17% | Mannitol 40% | dM-β-cyclodextrin |
| | | | 10% |
| | HPMC 17% | Trehalose 40% | HP-β-cyclodextrin |
| | | | 10% |
| | HPMC 17% | Trehalose 40% | β-cyclodextrin 10% |
| | HPMC 17% | Trehalose 40% | dM-β-cyclodextrin |
| | | | 10% |
| | HPMC 17% | Lactose 40% | HP-β-cyclodextrin |
| | | | 10% |
| | HPMC 17% | Lactose 40% | β-cyclodextrin 10% |
| | HPMC 17% | Lactose 40% | dM-β-cyclodextrin |
| | | | 10% |
| | Methylcellulose | Mannitol 40% | HP-β-cyclodextrin |
| | 17% | | 10% |
| | Methylcellulose | Mannitol 40% | β-cyclodextrin 10% |
| | 17% | | |
| | Methylcellulose | Mannitol 40% | dM-β-cyclodextrin |
| | 17% | | 10% |
| | Methylcellulose | Trehalose 40% | HP-β-cyclodextrin |
| | 17% | | 10% |
| | Methylcellulose | Trehalose 40% | β-cyclodextrin 10% |
| | 17% | | |
| | Methylcellulose | Trehalose 40% | dM-β-cyclodextrin |
| | 17% | | 10% |
| | Methylcellulose | Lactose 40% | HP-β-cyclodextrin |
| | 17% | | 10% |
| | Methylcellulose | Lactose 40% | β-cyclodextrin 10% |
| | 17% | | |
| | Methylcellulose | Lactose 40% | dM-β-cyclodextrin |
| | 17% | | 10% |
| | HPMC 20% | Mannitol 46% | Polysorbate 80 1% |
| | HPMC 20% | Mannitol 46% | Polysorbate 20 1% |
| | HPMC 20% | Trehalose 46% | Polysorbate 80 1% |
| | HPMC 20% | Trehalose 46% | Polysorbate 20 1% |
| | HPMC 20% | Lactose 46% | Polysorbate 80 1% |
| | HPMC 20% | Lactose 46% | Polysorbate 20 1% |
| | Methylcellulose | Mannitol 46% | Polysorbate 80 1% |
| | 20% | | |
| | Methylcellulose | Mannitol 46% | Polysorbate 20 1% |
| | 20% | | |
| | Methylcellulose | Trehalose 46% | Polysorbate 80 1% |
| | 20% | | |
| | Methylcellulose | Trehalose 46% | Polysorbate 20 1% |
| | 20% | | |
| | Methylcellulose | Lactose 46% | Polysorbate 80 1% |
| | 20% | | |
| | Methylcellulose | Lactose 46% | Polysorbate 20 1% |
| | 20% | | |
| | Chitosan 20% | Mannitol 47% | |
| | Chitosan 20% | Trehalose 47% | |
| | Chitosan 20% | Lactose 47% | |
| | HPMC 14% | Mannitol 33% | Dodecylphoshocholine |
| | | | 20% |
| | HPMC 14% | Trehalose 33% | Dodecylphoshocholine |
| | | | 20% |
| | HPMC 14% | Lactose 33% | Dodecylphoshocholine |
| | | | 20% |
| | Methylcellulose | Mannitol 33% | Dodecylphoshocholine |
| | 14% | | 20% |
| | Methylcellulose | Trehalose 33% | Dodecylphoshocholine |
| | 14% | | 20% |
| | Methylcellulose | Lactose 33% | Dodecylphoshocholine |
| | 14% | | 20% |

Abbreviations:
HP-β-cyclodextrin: hydroxypropyl-β-cyclodextrin
dM-β-cyclodextrin: dimethyl-β-cyclodextrin

### EXAMPLE 2

### Spray Drying Protocol

### Formulations:

API (ondansetron, metoclopramide or domperidone) with excipients exemplified in Table 1.

Use the hydrochloride salt of ondansetron, hydrochloride salt of metoclopramide and free base or maleate salt of domperidone.

### Preparation of Feed Solution:

Feed solutions prepared according to the details in Table 1. Dissolve the antiemetic API in an appropriate solvent. Prepare the excipient solution. Add the antiemetic API solution to the excipient solution in small aliquots until all components are combined.

### Spray Drying:

Feed solutions to be spray dried using a ProCepT fitted with an ultrasonic nozzle and using the parameters below:

TABLE 2
Spray drying conditions

| Inlet Temperature | Airflow In | | |
|---|---|---|---|
| (° C.) | (m³/min) | Cooling Air (Bar) | Feed Rate (g/min) |
| 100-110 | 0.66-0.72 | 0-0.50 | 1.6-2.2 |

Those skilled in the art will be familiar with translating these parameters to produce a powder using other models of spray drier e.g. a Niro spray drier, different nozzle types e.g. two-fluid nozzle or a pressure nozzle and at different scales.

Target a particle size suitable for nasal delivery (X₅₀—30-50 μm, X₁₀<10%). Spray dried powders were characterised by assay, particle size distribution, related substances and for residual moisture content.

### EXAMPLE 3

### Intranasal Antiemetic Preclinical Study

A proof-of-concept study was performed assessing the pharmacokinetics and antiemetic activity of an intranasal dry powder antiemetic formulation. The study was performed in a small animal model.

### Part 1: Pharmacokinetics

The animals received one of the following treatments:
1. Intranasal dry powder antiemetic
2. Commercial oral antiemetic
3. Commercial IV antiemetic
4. Intranasal dry powder placebo—negative control

Blood samples were taken at regular intervals, starting from relatively early time points, such as 5 minutes. The samples were processed and the concentration of the antiemetic measured using an appropriate analytical method. The pharmacokinetic parameters, such as Cₘₐₓ, Tₘₐₓ, AUC and T½ were reported. A suitable statistical analysis was conducted on the blood antiemetic concentrations and pharmacokinetic parameters to compare the results from each group.

### Part 2: Efficacy

The antiemetic activity was assessed in an emesis model, such as the ferret cisplatin emesis model, which is considered the standard for testing new antiemetics. Cisplatin triggers nausea and vomiting which can persist for a few hours.

The animals received one of the following treatments (same as Part 1):
1. Intranasal dry powder antiemetic
2. Commercial oral antiemetic
3. Commercial IV antiemetic
4. Intranasal dry powder placebo—negative control

The test treatments were assessed for their ability to reduce nausea and vomiting over the course of a few hours. The animals were kept under observation and parameters recorded such as the number of retching episodes, the number of vomiting episodes and the onset time to first emetic event for each treatment group. A suitable statistical analysis was conducted for each parameter to compare the results between groups.

### Outcomes

This study provided an early indication of how well the intranasal dry powder antiemetic formulation performs against currently approved products. The intranasal route was evaluated for its ability to allow drug absorption and induce behavioural effects.

1. A dry powder pharmaceutical composition for intranasal delivery, wherein the composition comprises a therapeutically effective amount of a medicament for the treatment of a disorder of the alimentary system, in free form or in salt form; one or more mucoadhesives; and optionally one or more pharmaceutically acceptable excipients.

2. A dry powder pharmaceutical composition according to claim 1 wherein the medicament for is an antiemetic medicament.

3. A dry powder pharmaceutical composition according to claim 1 wherein the medicament is for the treatment of a disorder of the gastrointestinal system.

4. A dry powder pharmaceutical composition according to claim 2 wherein the antiemetic medicament is selected from the group comprising 5-HT₃ receptor antagonists, dopamine antagonists, NK1 receptor antagonists, antihistamines, cannabinoids, benzodiazepines, anticholinergics and steroids, in free form or in salt form; and combinations thereof.

5. A dry powder pharmaceutical composition according to claim 2 wherein the antiemetic medicament is a 5-HT₃ receptor antagonist is a setron which is selected from one or more of dolasetron, granisetron, ondansetron, tropisetron, alosetron or palonosetron, in free form or in salt form or as a derivative thereof, and combinations thereof.

6. (canceled)

7. A dry powder pharmaceutical composition according to claim 5 wherein the 5-HT₃ receptor antagonist is ondansetron, in free form or in salt form or as a derivative thereof.

8. (canceled)

9. A dry powder pharmaceutical composition according to claim 1 wherein the medicament is a dopamine antagonist selected from one or more of amisulpride, domperidone, olanzapine, haloperidol, alizapride, prochlorperazine, chlorpromazine or metoclopramide, in free form or in salt form or as a derivative thereof; and combinations thereof.

10.-17. (canceled)

18. A dry powder pharmaceutical composition according to claim 1 wherein the composition is suitable for intranasal delivery using a powder inhaler, insufflator, powder sprayer or nebuliser.

19. A dry powder pharmaceutical composition according to claim 1 wherein primary particles in the dry powder composition will generally have a particle size distribution of D₅₀ of from about 20 to 60 μm.

20. A dry powder pharmaceutical composition according to claim 19 wherein the primary particles have a mean diameter >10 μm.

21. A dry powder pharmaceutical composition according to claim 1 wherein the number of particles with a diameter of 10 μm or less will be ≤20% w/w.

22. A dry powder pharmaceutical composition according to claim 1 wherein the one or more pharmaceutically acceptable excipients comprises a carrier and wherein the carrier is a sugar.

23. (canceled)

24. A dry powder pharmaceutical composition according to claim 22 wherein the carrier is selected from the group comprising one or more of glucose, saccharose, lactose and fructose, saccharides, disaccharides, sugar alcohols such as mannitol or sorbitol, lactose, lactitol, dextrates, dextrose, maltodextrin, saccharides including monosaccharides, disaccharides, polysaccharides; sugar alcohols such as arabinose, ribose, mannose, sucrose, trehalose, and maltose, dextran.

25. A dry powder pharmaceutical composition according to claim 24 wherein the carrier is selected from the group consisting of one or more of mannitol, trehalose and lactose.

26. A dry powder pharmaceutical composition according to claim 1 wherein the mucoadhesive is a mucoadhesive polymer, selected from an alkyl cellulose, a hydroxyalkyl cellulose, an alginate, a polyacrylic acid, gelatin, gellan gum, a polyethylene glycol, pectin; and combinations thereof.

27. A dry powder pharmaceutical composition according to claim 26 wherein the mucoadhesive is methylcellulose.

28. A dry powder pharmaceutical composition according to claim 26 wherein the mucoadhesive is hydroxypropyl methylcellulose (HPMC).

29. A dry powder pharmaceutical composition according to claim 1 wherein the composition includes one or more absorption enhancers.

30. A dry powder pharmaceutical composition according to claim 29 wherein the absorption enhancer is selected from cyclodextrins, phospholipids such as dodecylphosphocholine, surfactants, such as polysorbates and chitosan; and combinations thereof.

31. A dry powder pharmaceutical composition according to claim 5 wherein the setron is present as a pharmaceutically acceptable salt.

32.-44. (canceled)

45. A method of treating, preventing or alleviating a disorder of the alimentary system, said method comprising administering a therapeutically effective amount of a pharmaceutical composition in dry powder form, said composition comprising a medicament for the treatment of a disorder of the alimentary system, in free form or in salt form; one or more mucoadhesives; and optionally one or more pharmaceutically acceptable excipients.

46. A method according to claim 45 wherein the disorder of the alimentary system is emesis.

47. A method according to claim 45 wherein the disorder of the alimentary system is a gastrointestinal disorder.

48. (canceled)

49. A method according to claim 45 which comprises nasal administration of the pharmaceutical composition in dry powder form.

50. A method according to claim 45 wherein the medicament is a setron, in free form or in salt form or as a derivative thereof.

51. (canceled)

52. A method according to claim 50 wherein the setron is ondansetron, in free form or in salt form or a derivative thereof.

53.-62. (canceled)

63. A dry powder nasal inhaler containing a medicament for treating, preventing or alleviating a disorder of the alimentary system, said medicament being in dry powder form, in free form or in salt form or a derivative thereof, one or more mucoadhesives; and optionally one or more pharmaceutically acceptable excipients.

64.-72. (canceled)
